(19) United States
(12) Patent Application Publication
(10) Pub. No.: US 2021/0290333 A1
(43) Pub. Date: Sep. 23, 2021

(54) Medical Protective Device

(71) Applicant: Hillever Innovation Technology Corporation, New Taipei City (TW)

(72) Inventor: Han-Hui Lee, New Taipei City (TW)

(21) Appl. No.: 17/201,149

(22) Filed: Mar. 15, 2021

(30) Foreign Application Priority Data
Mar. 18, 2020 (TW) 109109059

(51) Int. Cl. A61B 90/00

(52) U.S. Cl. CPC A61B 90/05; A61B 2217/005

(57) ABSTRACT

A medical protective device is disclosed. The medical protective device includes a first cover and a second cover. The first cover includes a first plate and a negative pressure suction hole, wherein the first plate has a first hole. The second cover is adapted to be engaged under the first cover and to approach the mouth and the nose of a patient. The second cover includes a second plate and a plurality of extraction holes, wherein the second plate has a second hole in connection with the first hole. When the first cover and the second cover are engaged, a gap is present between the first cover and the second cover. Thus, infectious droplets produced by the mouth and the nose of the patient can be eliminated by the negative pressure suction hole through the plurality of extraction holes and the gap.

### BACKGROUND OF THE INVENTION

### Field of the Invention

The present invention relates to a medical protective device and, more particularly, to a medical protective device capable of reducing splashing and infection by droplets during medical behaviors conducted by medical staff.

### Description of the Prior Art

In current medical behaviors, medical staff are frequently required to be in close contact with patients. For example, when throat swabs, endotracheal intubation with laryngoscope, bronchoscopy, or other inspections or treatments of teeth and oral cavity are performed, a patient may produce contaminants such as saliva, body fluids, blood splashes, or vomit caused by physical conflict, resistance or discomfort. At this point in time, medical staff are extremely likely infected as being directly splashed by droplets or being in contact with infectious vomit. But so far, measures for effectively preventing the situations above are not present in the prior art.

Therefore, there is a need for a novel medical protective device that is safe and easy to use so as to resolve the drawbacks of the prior art.

### SUMMARY OF THE INVENTION

It is a primary object of the present invention to provide a medical protective device that achieves an effect of preventing infection of medical staff.

To achieve the object above, the medical protective device of the present invention is applicable to the face of a patient. The medical protective device includes a first cover and a second cover. The first cover includes a first plate and a negative pressure suction hole, wherein the first plate has a first hole. The second cover is adapted to be engaged under the first cover and to approach the mouth and the nose of a patient. The second cover includes a second plate and a plurality of extraction holes, wherein the second plate has a second hole. The second plate corresponds to the first plate, such that the second hole is in connection with the first hole. When the first cover and the second cover are engaged, a gap is present between the first cover and the second cover. Thus, infectious droplets produced by the mouth and the nose of the patient can be eliminated by the negative pressure suction hole through the plurality of extraction holes on the second cover and the gap between the two covers.

### BRIEF DESCRIPTION OF THE DRAWINGS

FIG. 1A is an exploded schematic diagram of a medical protective device according to a first embodiment of the present invention;

FIG. 1B is a bottom view of a second cover of the medical protective device according to the first embodiment of the present invention;

FIG. 2 is a three-dimensional schematic diagram of the medical protective device in assembly according to the first embodiment of the present invention;

FIG. 3 is a schematic diagram of the medical protective device according to the first embodiment of the present invention applied to a patient;

FIG. 4 is a cross-sectional diagram of a side of the medical protective device in use according to the first embodiment of the present invention;

FIG. 5 is a three-dimensional schematic diagram of a medical protective device according to a second embodiment of the present invention; and

FIG. 6 is a cross-sectional diagram of the medical protective device taken along A-A side in FIG. 5 according to the second embodiment of the present invention.

### DETAILED DESCRIPTION OF THE EMBODIMENTS

For the examiner to better understand the technical contents of the present invention, preferred embodiments are described below.

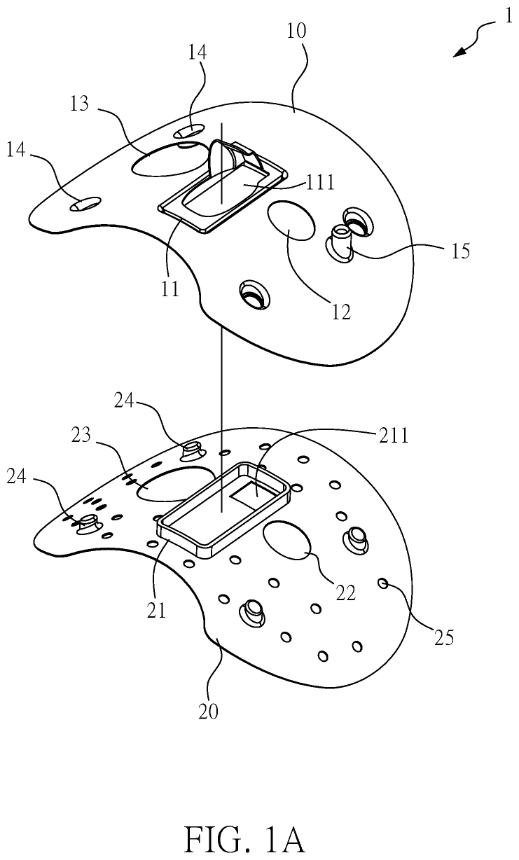

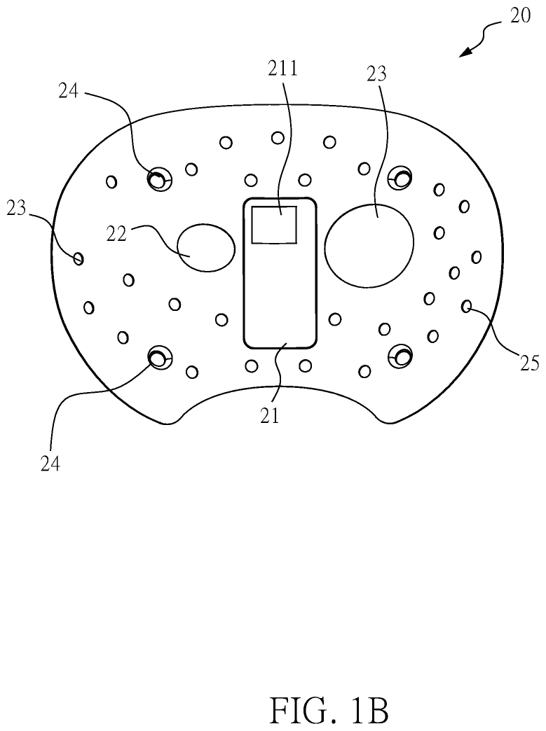

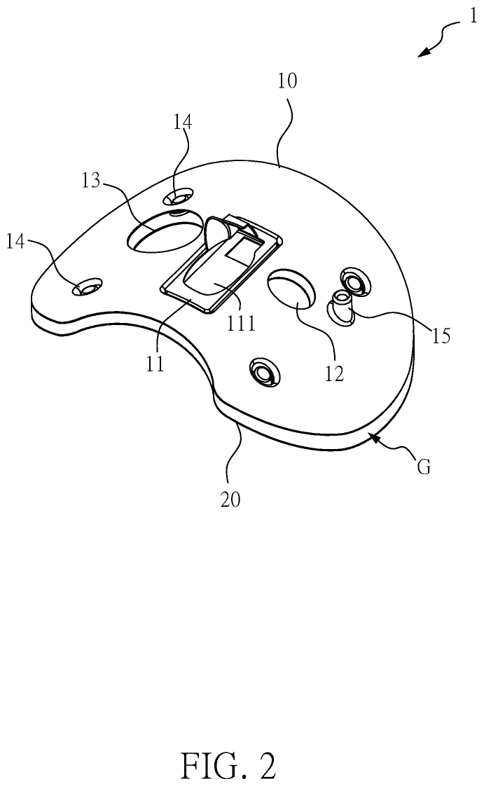

FIG. 1A shows an exploded schematic diagram of a medical protective device according to a first embodiment of the present invention. FIG. 1B shows a bottom view of a second cover of the medical protective device according to the first embodiment of the present invention. FIG. 2 shows a three-dimensional schematic diagram of the medical protective device in assembly according to the first embodiment of the present invention. It should be noted that, although terms such as “first” and “second” are used to describe various elements, these elements are not limited by these terms, and these terms are only used to distinguish one element from another element. For example; without departing from the scope of the various embodiments described, a first cover may be referred to as a second cover, and similarly; a second cover may be referred to as a first cover; the first cover and the second cover are both covers, but they are not the same.

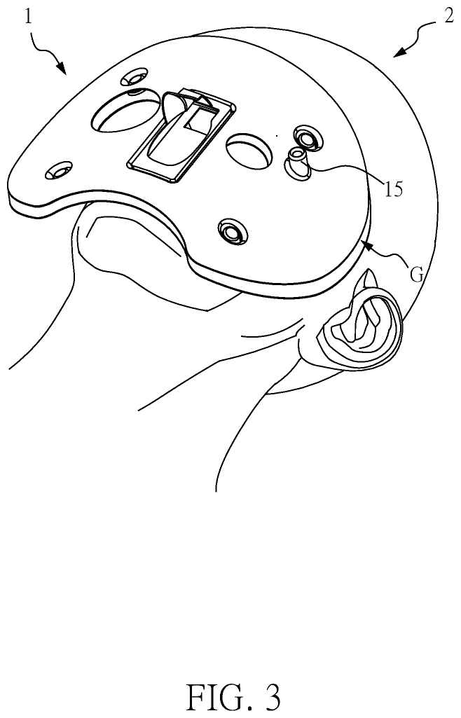

The present invention provides a medical protective device 1 adapted to cover the face of a patient 2 (as shown in FIG. 3), and is primarily acts as an auxiliary protective device, which is placed above the mouth and the nose of the patient 2 to provide blocking and suction functions when the patient 2 is in the process of receiving endotracheal intubation, so as to mitigate the risk of infection of medical staff directly exposed to highly infectious droplets splashed by the patient 2 during the intubation process. However; the medical protective device 1 of the present invention is not limited to being used as a protection measure for medical staff during the intubation process. The medical protective device 1 includes a first cover 10 and a second cover 20, The first cover 10 and the second cover 20 may be made of a transparent acrylic material; however, the present invention is not limited to such material. The second cover 20 is adapted to be engaged with the first cover 10 to locate under the first cover 10 and to approach the mouth and the nose of the patient 2. The first cover 10 may include a first plate 11, a third hole 12, a fifth hole 13, a plurality of first engaging structures 14 and a negative pressure suction hole 15. The second cover 20 includes a second plate 21, a fourth hole 22, a sixth hole 23, a plurality of second engaging structures 24 and a plurality of extraction holes 25. The negative pressure suction hole 15 may be connected to a vacuum suction machine (not shown) to extract air. Moreover, the suction force near the negative pressure suction hole 15 is larger and the suction force away from the negative pressure suction hole 15 is smaller, so the number of the plurality of extraction holes 25 closer to the negative pressure suction hole 15 of the first cover 10 on the second cover 20 is less than the number of the plurality of extraction holes 25 away from the negative pressure suction hole 15, so as to provide a better suction effect. It should be noted that, the present invention does not limit that the first cover 10 and the second cover 20 have to include all of the elements above, nor does it limit that the first cover 10 and the second cover 20 can include only the elements above.

The first plate 11 of the first cover 10 corresponds to the second plate 21 of the second cover 20, and the first plate 11 and the second plate 21 respectively have a first hole 111 and a second hole 211, wherein the first hole 111 is in communication with the second hole 211 when the first cover 10 and the second cover 20 are assembled. In one embodiment of the present invention, the first cover 10 and the second cover 20 can be engaged with each other by coupling structures of the first plate 11 and the second plate 21, such that the first cover 10 and the second cover 20 are not directly adhered to each other, and a gap G is present between the first cover 10 and the second cover 20. In this embodiment, the first cover 10 and the second cover 20 can also be engaged with each other by the plurality of first engaging structures 14 and the plurality of second engaging structures 24. The first engaging structures 14 and the second engaging structures 24 can be protruding structures corresponding to each other, and allow the gap G to be kept between the first cover 10 and the second cover 20.

Moreover, the third hole 12 of the first cover 10 corresponds to the fourth hold 22 of the second cover 20, and the fifth hole 13 of the first cover 10 corresponds to the sixth hole 23 of the second cover 20, thereby forming holes in connection with each other. Sizes of the fifth hole 13 and the sixth hole 23 and sizes of the third hole 12 and the fourth hole 22 may be different. For example, in this embodiment, the sizes of the fifth hole 13 and the sixth hole 23 are greater than the sizes of the third hole 12 and the fourth hole 22. The fifth hole 13 and the sixth hole 23 may be located on one side of the first hole 111 and the second hole 211, and the third hole 12 and the fourth hole 22 may be located on the other side of the first hole 111 and the second hole 211; however, the present invention is not limited to the structure above.

Refer to FIG. 3 showing a schematic diagram of the medical protective device according to the first embodiment of the present invention applied to a patient.

Both the first cover 10 and the second cover 20 are shaped to have an arc for adapting to the facial dimensions of the patient 2. Sizes of the first cover 10 and the second cover 20 may also be designed according to the age of the patient 2, and the present invention does not limit the sizes of the first cover 10 and the second cover 20.

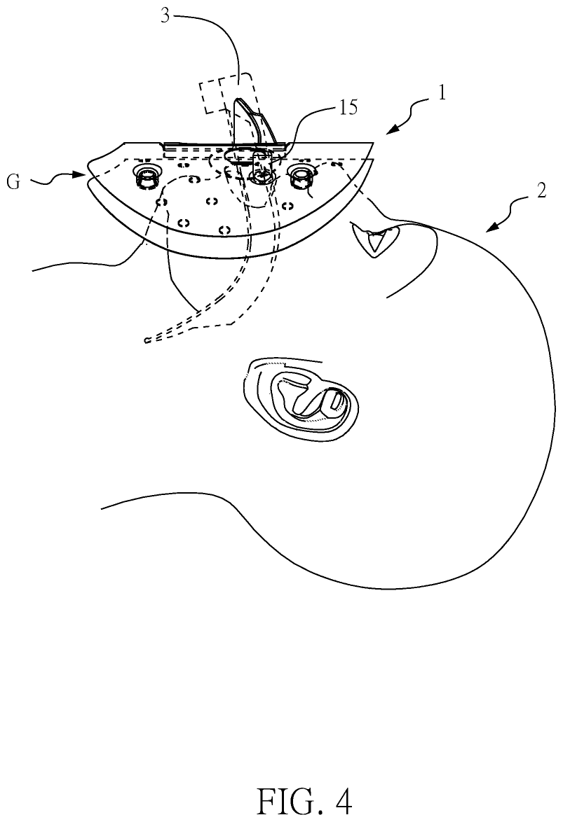

Refer to FIG. 4 showing a cross-sectional diagram of a side of the medical protective device in use according to the first embodiment of the present invention.

In this embodiment, the sizes of the first hole 111 and the second hole 211 allow a laryngoscope 3 to pass through; however, the present invention is limited to being applied to the laryngoscope 3, and the first plate 11 and the second plate 21 may also be replaced with specifications of other medical supplies used by medical staff, or be used for treatments and inspections of the oral cavity and teeth. Furthermore, a channel formed by the third hole 12 and the fourth hole 22 and a channel formed by the fifth hole 13 and the sixth hole 23 may be provided with medical supplies, and the present invention does not limit the types of the medical supplies. The negative pressure suction hole 15 is connected to the vacuum suction machine. Minute contaminating droplets splashed from the respiratory tract of the patient 2 and those in the air are distributed under the second cover 20, or may be dispersed through the plurality of extraction holes 25 of the second cover 20 to the gap G between the first cover 10 and the second cover 20. Thus, the infectious droplets such as saliva, body fluids or blood produced by the mouth and the nose of the patient 2 during a medical treatment can be extracted and eliminated by the negative pressure suction hole 15 in coordination with the plurality of extraction holes 25 on the second cover 20. The infectious droplets distributed in the gap G between the first cover 10 and the second cover 10 are also eliminated by the negative pressure suction hole 15, Moreover, in the presence of a large amount of splatter, the infectious droplets that are not timely extracted away by the negative pressure suction hole 15 can also be discharged downward through the gap G. If vomit is produced, the vomit is not splashed onto medical staff but can be discharged downward through the gap G. Thus, medical staff does not come into direct contact with the infectious droplets of the patient 2, hence protecting medical staff from infection.

It should be noted that, a film that can be easily replaced may be provided between the first cover 10 and the second cover 20, wherein the film can be electrostatic cotton; however, the present invention is not limited thereto. Accordingly, even better protection effects are achieved, and durability of the medical protective device 1 can also be increased by means of replacing the film.

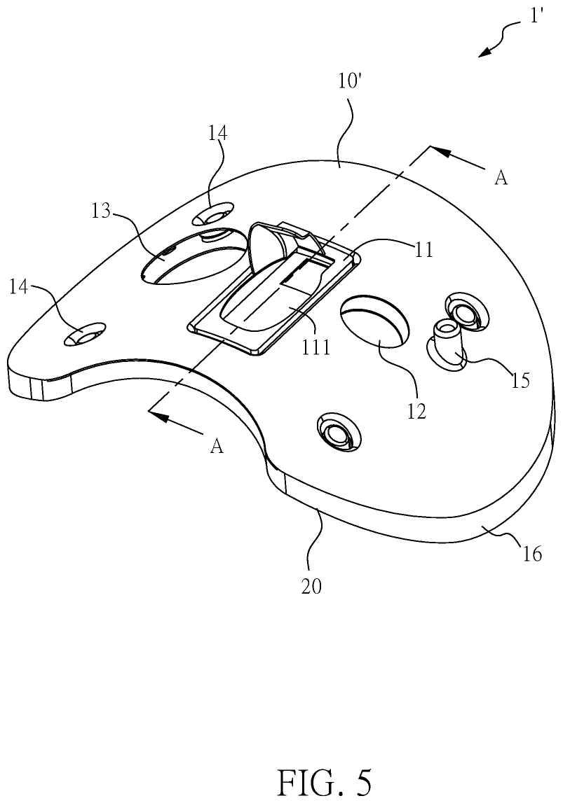

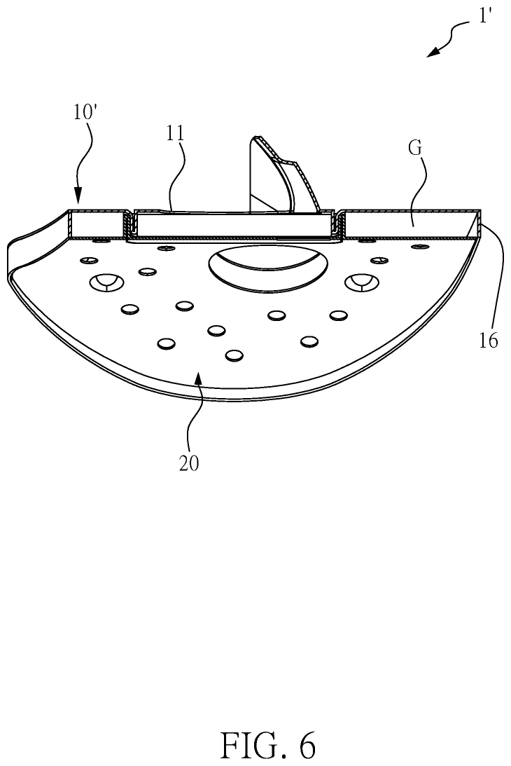

Refer to FIG. 5 showing a three-dimensional schematic diagram of a medical protective device according to a second embodiment of the present invention. FIG. 6 shows a cross-sectional diagram of the medical protective device taken along A-A side in FIG. 5 according to the second embodiment of the present invention.

In the second embodiment of the present invention, a first cover 10′ of a medical protective device 1′ can further include a sidebar 16. When the first cover 10′ and the second cover 20 are engaged, the sidebar 16 is in contact with an edge of the second cover 20. A gap G is present between the first cover 10′ and the second cover 20, but the gap G is not exposed. Thus, infectious droplets produced during a medical treatment are completely enveloped between the first cover 10′ and the second cover 20, achieving even better protection effects.

It should be noted that, the disclosure above is merely embodiments and is not limited to these embodiments. For example, other embodiments without departing from the fundamental structure of the present invention are to be encompassed within the protection scope of the patent rights of the present invention, and are to be defined by the appended claims.

What is claimed is:

1. A medical protective device, suitable for a face of a patient, the medical protective device comprising:
a first cover, comprising a first plate and a negative pressure suction hole, the first plate having a first hole; and
a second cover, adapted to be engaged under the first cover and to approach the mouth and the nose of a patient, the second cover comprising a second plate and a plurality of extraction holes, the second plate having a second hole, the second plate corresponding to the first plate such that the second hole is in connection with the first hole;
wherein, when the first cover and the second cover are engaged, a gap is present between the first cover and the second cover, and infectious droplets produced by the mouth and the nose of the patient are eliminated by the negative pressure suction hole through the plurality of extraction holes on the second cover and the gap between the two covers.

2. The medical protective device according to claim 1, wherein the first cover further comprises a third hole and the second cover further comprises a fourth hole, wherein the fourth hole corresponds to the third hole.

3. The medical protective device according to claim 2, wherein the first cover further comprises a fifth hole and the second cover further comprises a sixth hole, wherein the sixth hole corresponds to the fifth hole.

4. The medical protective device according to claim 3, wherein the fifth hole and the sixth hole are located on one side of the first hole and the second hole, and the third hole and the fourth hole are located on the other side of the first hole and the second hole.

5. The medical protective device according to claim 4, wherein sizes of the fifth hole and the sixth hole are greater than sizes of the third hole and the fourth hole.

6. The medical protective device according to claim 1, wherein the number of the plurality of extraction holes near the negative pressure suction hole is less than the number of the plurality of extraction holes away from the negative pressure suction hole.

7. The medical protective device according to claim 1, wherein the first cover further comprises a plurality of first engaging structures, the second cover further comprises a plurality of second engaging structures, and the first cover and the second cover are engaged with each other by the plurality of first engaging structures and the plurality of second engaging structures, such that the gap is kept between the first cover and the second cover.

8. The medical protective device according to claim 1, wherein the first cover and the second cover are engaged with each other by coupling structures of the first plate and the second plate, such that the gap is kept between the first cover and the second cover.

9. The medical protective device according to claim 1, wherein the first cover and the second cover are shaped to have an arc for adapting to facial dimensions of the patient.

10. The medical protective device according to claim 1, wherein the first cover and the second cover are applicable to uses of different medical supplies by replacement of different first plate and different second plate.

11. The medical protective device according to claim 1, wherein the first cover further comprise a first sidebar, and the sidebar is in contact with an edge of the second cover when the first cover and the second cover are engaged.

12. The medical protective device according to claim 1, wherein a film is further provided between the first cover and the second cover.

13. The medical protective device according to claim 1, wherein the negative pressure suction hole is connected to a vacuum suction machine.

14. The medical protective device according to claim 1, wherein the infectious droplets produced by the patient and eliminated by the negative pressure suction hole comprise saliva, body fluids and blood.
